(19) United States
(12) Patent Application Publication
(10) Pub. No.: US 2007/0265617 A1
(43) Pub. Date: Nov. 15, 2007

(54) DILATION CATHETER ASSEMBLY WITH BIPOLAR CUTTING ELEMENT

(75) Inventors: Zoran Falkenstein, Rancho Santa Margarita, CA (US); Boun Pravong, Corona, CA (US); Charles C. Hart, Summerville, SC (US); John R. Brustad, Dana Point, CA (US); Eric Nguyen, Corona, CA (US)

Correspondence Address: APPLIED MEDICAL RESOURCES CORPORATION, 22872 Avenida Empresa, Rancho Santa Margarita, CA, 92688, US

(73) Assignee: APPLIED MEDICAL RESOURCES CORPORATION, Rancho Santa Margarita, CA (US)

(21) Appl. No.: 11/748,955

(22) Filed: May 15, 2007

Related U.S. Application Data
(60) Provisional application No. 60/747,263, filed on May 15, 2006.

(51) Int. Cl. A61B 18/14

(52) U.S. Cl. 606/48

(57) ABSTRACT

A method and apparatus for the dilatation of obstructed body vessels, orifices and conduits, combined with a controlled and regulated incision of the dilated body vessels using a bipolar or quasi-bipolar electrosurgical cutting wire arrangement is presented.

### CROSS-REFERENCE TO RELATED APPLICATION

This application claims benefit of U.S. Provisional Application No. 60/747,263, filed May 15, 2006, the entire disclosure of which is hereby incorporated by reference as if set forth in full herein.

### FIELD OF THE INVENTION

The present invention relates generally to the field of electrosurgical devices and more specifically to a dilatation catheter having an expandable member comprising a cutting element that concurrently incises body tissue in a bipolar or quasi-bipolar fashion.

### BACKGROUND

Dilatation catheters are used to dilate body vessels, orifices and conduits, such as a constricted or obstructed ureter or urethra. Typically, a dilation catheter comprises an elongated catheter having an inflatable balloon at or near the distal end. A guide wire or other axial support means is often included to improve the ability to position the apparatus appropriately, usually visualized under fluoroscopy.

Dilation catheters incorporating an electrosurgical wire are described in U.S. Pat. Nos. 5,628,746 and 5,904,679, both issued to Clayman, on May 13, 1997 and May 18, 1999, respectively, both incorporated by reference in their entireties Clayman describes an electrosurgical cutting wire mounted longitudinally along the outer surface of the balloon. After correct positioning and inflation of the balloon, application of electrosurgical energy to the wire provides a clean, sharp incision in the tissue. This is accomplished by passing high frequency electrosurgical current from the active cutting electrode through the patients' tissue into a return electrode. This process cuts tissue in close proximity to the active electrode since the current density is high, while dispersion of the current towards the return electrode very quickly reduces the generation of heat within the tissue.

Despite the success of the monopolar electrosurgery technique, a few problems may arise during its use. For example, in some circumstances there may be a failure to cut. In order for an electrosurgical cutting event to take place, the electrode needs to be distanced from the tissue to be cut by a small amount, approximately 0.1 mm, to create a spark gap. In the monopolar configuration, a surgeon must allow sufficient time after applying current to heat enough tissue to create this gap before inflating the balloon. If the surgeon starts to inflate the balloon too quickly before the current is applied, the wire will imbed itself into the tissue and the current will simply pass from the wire into the patient with no cutting event.

A second reason a monopolar device may fail to cut is due to the use of saline or contrast in the urinary system, for example. Urine, saline, and contrast, used to highlight structures during fluoroscopy, all conduct electricity to some degree. If the amount of conductivity is high enough, the fluids in the urinary system around the monopolar device will conduct the electricity away through the urinary system and to the return electrode.

Also, in this monopolar electrosurgery configuration, a significant fraction of the total supplied energy is dissipated by the tissue between the return electrode and the active cutting electrode, far away from the active cutting site. In addition, stray current may have unintended tissue effects, not readily apparent to the physician. The human body is far from a homogenous conductor. Blood in arteries and veins can be much more conductive than the surrounding tissue and can be overheated in the process of conducting that electricity.

Finally, with monopolar electrosurgical devices, problems may arise if the surgeon activates the device in the patient without making contact with tissue. This can cause what is known as capacitive coupling and can cause another metallic component nearby to conduct electricity to tissue and cause a burn that is not known by the surgeon.

It would, therefore, be beneficial to provide an improved electrosurgical device which addresses the concerns listed above.

### SUMMARY OF THE INVENTION

The presented invention provides a method and apparatus to overcome the drawbacks of a monopolar cutting arrangement by supplying electrical energy in a bipolar or quasi-bipolar fashion. Unlike a monopolar arrangement, in a bipolar or quasi-bipolar configuration, the electrical current will flow only through tissue between closely-spaced electrodes, resulting in improved cutting, while reducing or eliminating stray current and capacitive coupling.

The bipolar or quasi-bipolar configuration does not require a return electrode pad on the patient's skin, thus eliminating any risk of return electrode burns. Instead, the active electrodes are placed on the surgical tool in close proximity of the tissue to be affected, thereby reducing the amount of tissue exposed to electrical energy in general. In this new inventive configuration, the device has a “built-in” spark gap between the two electrodes, thus producing more efficient cutting.

Importantly, the close proximity of the active and return electrode eliminates the risk of inadvertent tissue burns. Since the electrical energy only travels between the two electrodes, only the tissue between the two electrodes is affected and inadvertent tissue damage, outside of the surgeon's field of view, can be eliminated.

Thus, one embodiment of the present invention is directed to a bipolar dilation-and-cutting catheter assembly adapted for insertion into a body conduit of a patient, comprising an elongate tubular body having an axis and a distal end carrying a generally cylindrical radially dilatable member adapted to be positioned longitudinally in a body conduit and having properties for dilating generally radially of the tubular body; at least two wires carried by the tubular body exteriorly of the dilatable member, the wires disposed longitudinally of the tubular body and movable radially in a plane including the axis of the tubular body; means for dilating the dilatable member to exert dilation forces on the body conduit and to move the wires to a position adjacent to the tissue of the body conduit; and means for activating the wires to create an incision in the tissue.

Another embodiment of the present invention is directed to a bipolar dilation catheter assembly adapted for insertion into a body conduit of a patient, comprising an elongate tubular body having an axis and a distal end carrying a generally cylindrical inflatable balloon that is adapted to be connected to a source of inflation fluid and that is adapted to be positioned longitudinally in a body conduit and having properties for dilating generally radially of the tubular body; a pair of wires carried by the tubular body exteriorly of the inflatable balloon, the wires disposed longitudinally of the tubular body and movable radially in a plane including the axis of the tubular body, wherein at least one of the wires is adapted to connect to a generator having electrical power to create a current density in the tissue proximate to the wires, the electrical power being sufficient to cut the tissue.

Still another embodiment of the present invention is directed to a quasi-bipolar dilation-and-cutting catheter assembly adapted for insertion into a body conduit of a patient, comprising an elongate tubular body having an axis and a distal end carrying a generally cylindrical radially dilatable member adapted to be positioned longitudinally in a body conduit and having properties for dilating generally radially of the tubular body; a first electrode, comprising a wire carried by the tubular body exteriorly of the dilatable member, the wire disposed longitudinally of the tubular body and movable radially in a plane including the axis of the tubular body; a second electrode disposed exteriorly around the surface of the dilatable member; means for dilating the dilatable member to exert dilation forces on the body conduit and to move the first and second electrodes to a position adjacent to the tissue of the body conduit; and means for activating the electrodes to create an incision in the living tissue.

Another embodiment of the present invention is directed to a quasi-bipolar dilation catheter assembly adapted for insertion into a body conduit of a patient, comprising an elongate tubular body having an axis and a distal end carrying a generally cylindrical inflatable balloon that is adapted to be connected to a source of inflation fluid and that is adapted to be positioned longitudinally in a body conduit and having properties for dilating generally radially of the tubular body; an electrode disposed around the outside surface of the balloon; and a wire carried by the tubular body exteriorly of the balloon, the wire disposed longitudinally of the tubular body and movable radially in a plane including the axis of the tubular body, wherein the wire is adapted to connect to a generator having electrical power to create a current density in the tissue proximate to the wire, the electrical power being sufficient to cut the tissue.

Yet another embodiment of the present invention is directed to an apparatus for cutting a body conduit, comprising a supporting structure having an outer surface; a first electrode having a first portion disposed in a fixed relationship with the supporting structure and a second portion disposed outwardly of the outer surface in a movable relationship with the supporting structure; moving means disposed between the supporting structure and the second portion of the first electrode for moving the second portion of the first electrode into proximity with the tissue to be cut; a second electrode, disposed on the exterior of the moving means; and activating means for electrically activating the electrodes to cut the body conduit,

### DESCRIPTION OF THE DRAWINGS

The present invention may best be understood by reference to the following description, taken in connection with the accompanying drawings in which the reference numerals designate like parts throughout the figures thereof and wherein.

FIG. 1a shows a schematic of a distal end of conventional dilatation balloon arrangement; FIG. 1b shows a cross-section through the balloon portion.

FIGS. 2a and 2b depicts a prior art dilatation balloon arrangement with a monopolar electrosurgical cutting wire arrangement, having a distal dilatation balloon, a proximal hand-piece and a multi-lumen tubing connecting the balloon with the hand-piece; FIG. 2c is a drawing showing two views of the balloon arrangement of FIGS. 2a and 2b.

FIG. 3 is a schematic of a prior art monopolar electrosurgical catheter arrangement showing the current traveling from a region of high current density to a region of very low current density.

FIG. 4 shows a prior art monopolar electrosurgical catheter arrangement showing risk of tissue burning by an increased current density at the site of a constriction.

FIGS. 5a and 5b show a bipolar electrosurgical catheter arrangement having two wire electrodes on the outside of a dilatation balloon.

FIGS. 6a and 6b show a quasi-bipolar electrosurgical catheter arrangement having one cutting wire electrode and a return electrode on the entire outside surface of a dilatation balloon.

FIGS. 7a and 7b show close-ups of insulation sleeves around two wire electrodes in a bipolar electrosurgical catheter arrangement; FIG. 7c shows a close-up of an insulation sleeve around a cutting wire electrode on a quasi-bipolar electrosurgical catheter arrangement.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the structures and/or methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following description is provided to enable any person skilled in the art to make and use the invention and sets forth the best modes contemplated by the inventors of carrying out their invention. Various modifications, however, will remain readily apparent to those skilled in the art, since the general principles of the present invention have been defined herein specifically to provide a method and apparatus for the combined dilatation and bipolar electrosurgical dissection of tissue.

To better understand the benefits and advantages of the invention in comparison to prior-art systems, we will describe the combined dilatation and electrosurgical cutting process in more detail.

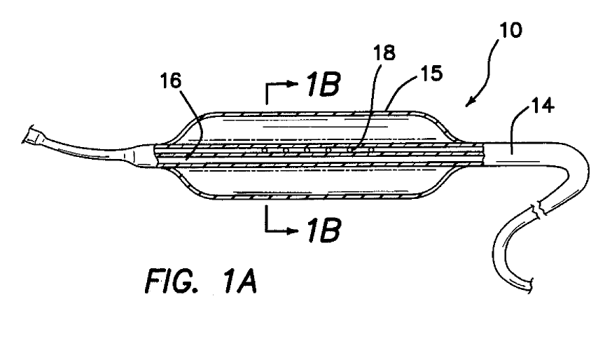

FIG. 1 depicts the distal end of a conventional dilatation catheter assembly, generally designated 10, that may be used for dilating a body vessel or conduit for treating a blockage or other obstruction, such as a catheter or urethra. The main elements of catheter assembly 10 are: a catheter body 14, having a double lumen and an inflatable balloon 15. A stiffening guide wire or stylet 16 extends longitudinally within one of the two inner catheter body lumens, facilitating guidance of the dilatation catheter assembly during insertion into a body conduit vessel or orifice towards an obstruction site. Once the catheter is correctly positioned, the body vessel can be dilated by inflating the balloon by pressurizing it with a fluid through the second lumen of the catheter body. The supply/drainage of fluid is realized by providing the distal end of the catheter body with a series of supply/drain holes 18, connecting the balloon to the second lumen of the catheter assembly 10.

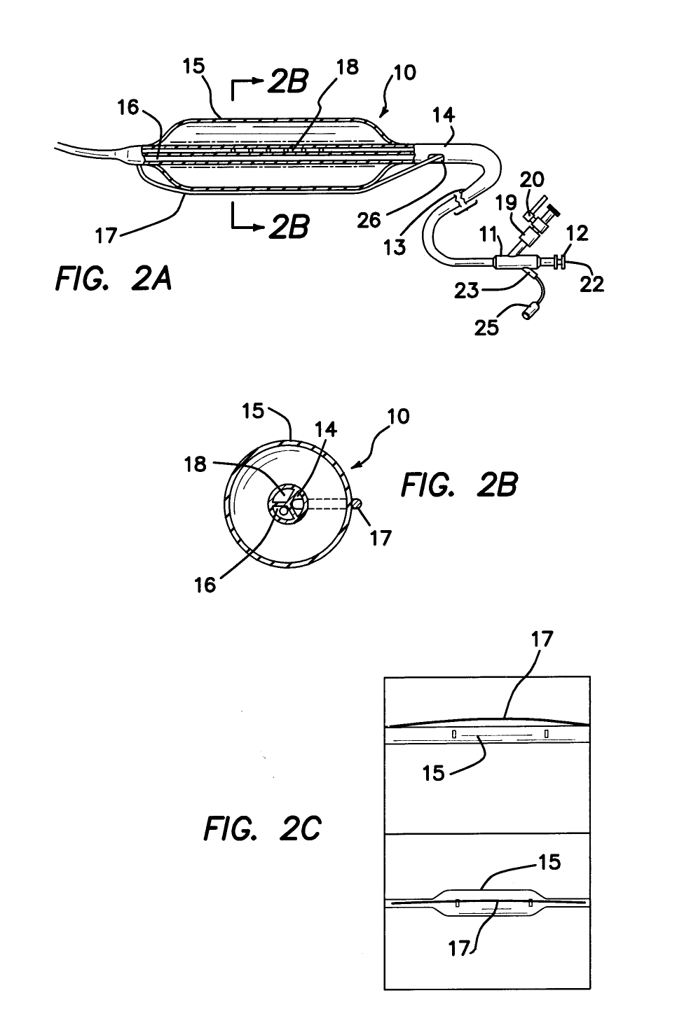

A dilatation catheter assembly with (monopolar) electrosurgical cutting element is schematically shown in FIGS. 2a and 2b; a drawing showing two views of such a catheter assembly is shown in FIG. 2c. As with the dilatation catheter assembly shown in FIG. 1, the main components are a catheter body 14, this time with a three-lumen configuration; an inflatable balloon member 15; a stiffening guide or stylet 16; and a cutting element or electrode 17, preferably activated by a radiofrequency electrosurgical cutting power source. An adapter 11 defines the proximal end 12 of the assembly 10 and provides a site for various ports to the assembly 10. As illustrated in FIGS. 2a and 2b, one of the three inner lumens serves as an inflation/deflation passageway 18, the second lumen carries the guide wire or stylet 16 and serves as a drainage/infusion passageway, and a third lumen carries the cutting element 17.

The adapter 11 serves as a site for a balloon inflation/deflation port 19 that is attached to a source of inflation medium (not shown) for inflating the balloon 15, or to a suction source (not shown) for deflating the balloon 15. Port 19 has a valve 20 for regulating the inflation medium or suction, as required. Port 19 connects into the proximal end of an inflation/deflation passageway 18 that extends from the port 19 to the inflatable balloon 15. The adapter 11 also serves as a site for the drainage tube inlet/outlet port 22 and a cutting element port 23. The drainage port 22 is connected to the proximal end of the lumen that carries the guide wire or stylet 16. The drainage port 22 may serve as a site for removing fluid from the lumen or as a site for infusing fluid into the lumen.

The distal end of the catheter body has a series of drain holes 18 to facilitate flushing the lumen with fluid or voiding the balloon 15. A “banana plug” cutting element connector 25 is affixed to the end of the cutting element port. The cutting element 17 extends from the connector 25 through the lumen of the catheter body 14, exits therefrom via an aperture 26, and continues along the exterior of the balloon 15.

The inflatable balloon 15 is preferably of the non-distensible variety, i.e., it can, when expanded, assume only a specific size and shape. Thus, the balloon member 15 cannot extend or bulge longitudinally within the body conduit beyond its predetermined diameter or length. Unlike elastic or elastomeric balloons, it must exert the inflation force radially against the enclosing body conduit or the like. In contrast, if an elastic or elastomeric balloon is expanded within the narrowed or constricted body conduit, the balloon will simply elongate rather than acting radially against the constriction. One material suitable for the balloon is low density polyethylene (LDPE).

In addition, however, the inflatable balloon preferably can maintain a constant temperature, even when current is passing through the cutting element. LDPE balloons alone may not maintain a constant temperature under these conditions. Accordingly, the LDPE balloon can be covered with a second balloon made from a material, such as silicone, which can withstand high temperatures (i.e., temperatures generated during electrosurgical cutting) and protect the LDPE from bursting during the heating process. This balloon-within-a-balloon arrangement provides both the non-distensible qualities and the temperature profile desired for use with a cutting element as described above.

The electrosurgical cutting element 17 is in the nature of a wire that extends generally parallel to the longitudinally extending inflatable balloon 15. The material used for the wire can be any kind of materials currently used for electrosurgical cutting. For example, the wire can be made of stainless steel or tungsten. The wire is encapsulated in an electrical insulation sheet, with an external incising edge that exposes the wire outwardly from the balloon member. Alternatively, the cutting element 17 may be a sharp-edged or a cutting element activatable by a radiofrequency power source.

The portion of the exterior of the inflatable balloon 15 that is exposed to the cutting element 17 may carry a protective cover (not shown) to further guard against the inflatable balloon 15 being incised by the cutting element 17. The cutting element 17 may be carried at a predetermined spacing from the balloon surface or directly on the surface. When carried on the surface the cutting element 17 may be an integral part of the surface or may be attached to the surface. In one embodiment, the cutting element 17 is manually extendable or retractable via the connector 25 into and out of the catheter body 14.

In some embodiments, the cutting element 17 is disposed parallel to the balloon 15. With inflation of the balloon within a body conduit, the inflation causes the cutting element 17 to move radially outward until the cutting element contacts the surrounding tissue. Continued radial expansion of the balloon 15 causes the balloon to exert pressure on the tissue, subjecting the tissue to a substantially uniform tangential tension. Then, a radiofrequency current can be passed through the cutting element 17.

This combined cutting and dilating action expands the tissue without building up excess stress within the tissue that can lead to tearing. Instead, the tissue is electrosurgically cut in a clean, concentrated, generally longitudinally fashion by the cutting element 17, without the dilatation causing uncontrollable tearing of the tissue and excessive trauma and bleeding. The process of electrosurgical incision is visualized under fluoroscopy and is witnessed by a full dilatation of the balloon.

After the vessel, conduit, or orifice is incised and dilated, and the blockage or obstruction is relieved, the power through the radiofrequency cutting element 17 is discontinued. The inflated balloon 15 now provides the additional benefit of acting as a tamponade to reduce bleeding. If desired, the cutting element 17 can be retracted prior to complete deflation of the balloon, and the balloon may be left in place to act as tampon. Then the balloon can be deflated by operation of the inflation/deflation port valve and retracted out of the body conduit or orifice.

Monopolar dilatation catheter assemblies are described in detail in U.S. Pat. Nos. 5,628,746 and 5,904,679, both to Clayman, both of which are hereby incorporated by reference in their entireties. One example of a monopolar dilatation catheter assembly arrangement comprises a 0.015-inch stainless steel cutting wire, 0.0035-inch fluorinated ethylene propylene (FEP) wire insulation, a low density polyethylene (LDPE) balloon with 0.0015-inch wall thickness surrounded by a silicone balloon with approximately 0.0025-inch wall thickness when inflated (or approximately 0.004-inch when non-inflated). Typically, the outer diameter of the inflated balloon(s) is approximately 24 French.

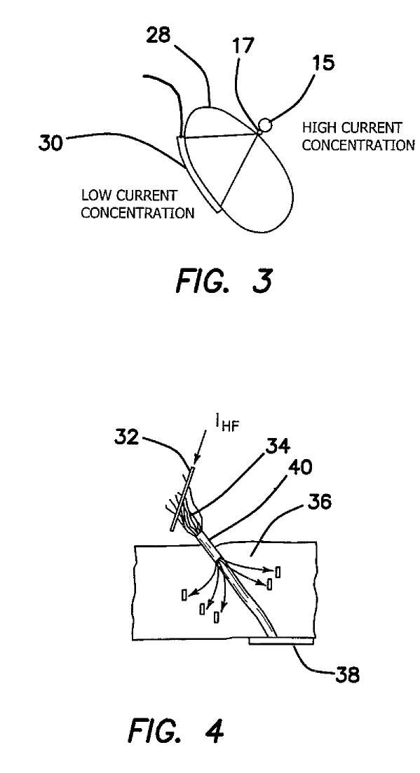

The described monopolar cutting process is schematically depicted in FIG. 3. Here, the dilatation balloon 15 is shown in inflated condition, pressing the electrosurgical cutting element 17 against the tissue to be dissected 28 as described in previous section. The opposing electrode to the cutting wire is the return electrode patch 30, which is firmly attached to the patient's skin. The electrical circuit between cutting wire element and the return electrode composes of the entire tissue between the two electrodes, which includes—but is not limited to—the tissue in immediate contact and proximity to the cutting wire element.

As the cutting wire element is applied with electrical power from an electrosurgical power supply, the electrical current will flow from the exposed wire section of the cutting element 17 to the tissue in immediate contact to the wire. From there, the same amount of electrical current will quickly disperse within the surrounding tissue towards the return electrode path, where it is collected and returned to the electrosurgical generator.

In terms of electrosurgical processing, the only noticeably affected area during this process is the tissue in immediate contact and very close proximity to the exposed cutting wire element. Here, both the voltage drop and current density are high (and eventually lead to the formation of an electrical arc), whereas in the remaining bulk of the tissue towards the return electrode both the voltage drop and current density are low. In other words, the energy deposited into the tissue is very high in density in close proximity to the cutting wire, whereas the energy density in the remaining bulk tissue is very low. As a result, the very high energy density in the tissue close to the cutting wire leads to quick evaporation of the tissue (electrosurgical cutting), while the very low energy density in the remaining bulk tissue towards the return electrode merely causes an insignificant raise in tissue temperature. The transition region of moderate energy density is in immediate contact to the evaporated tissue, and expands maximally to a few millimeters into the bulk tissue. In electrosurgical processing, this region is also referred to as the “thermal spread”.

Even though the electrosurgical effect in the bulk tissue—where the energy density is very low—is insignificant to practically non-existent, there is still a considerable amount of total energy deposited in the tissue overall. While is this is not a problem per se as long as the total energy is distributed in the bulk tissue, it allows the possibility of two failure modes. Both risks are based on an inadvertent increase of the current density (and thereby energy density) within the bulk tissue, as the electrical power is traveling towards the return electrode.

The first failure mode occurs when the return electrode partially delaminates from the patients' skin tissue, resulting in a reduction of the contact area. This in turn will increase the current density (and energy density) at the contact area between return electrode and the patients' skin. Instead of the electrical current continuously dispersing through the bulk tissue towards the return electrode, a delaminating return electrode results in the electrical current concentrating again when reaching the return electrode patch. If the energy density is high enough, this can lead to severe burns of the patient's skin. Most modern return electrode patches use strong, electrically conductive adhesives that firmly attach to the patient's skin, as well as a “split” electrode arrangement that allows the ESU to monitor that the entire return electrode patch is firmly connected to the patients' skin. Nonetheless, the possibility of delamination, however minimal, poses ad hoc some risk as current is traveling through large volumes of tissue.

The second failure mode is similar in principle and occurs when the monopolar electrical current flows through constrictions in the tissue as it travels through the bulk tissue towards the return electrode. This is illustrated in FIG. 4, showing the cutting element 32 pressed firmly against the tissue 34 to be cut. Instead of the electrical current dispersing throughout the bulk tissue 36 as it travels towards the return electrode 38, a constriction in the cross section of a tissue segment 40 will exhibit an increase in current density. If the resulting current density (i.e., energy density) is high enough, this can lead to severe burns, or even cuts of the constricted tissue. This failure mode is of particular concern as it can occur outside of the surgeon's view.

Other potential problems with monopolar electrosurgical cutting devices have been discussed above. For example, capacitive coupling, resulting from activation of a monopolar electrosurgical cutting device without making contact with tissue, may lead to unintended burns where another metallic component nearby conducts electricity away from the intended cutting site. Cutting may fail in the presence of saline, urine, contrast solution or other conductive solutions, and training is required to ensure that surgeons apply current before expanding the balloon to create a spark gap for efficient cutting. The invention described herein, a bipolar or quasi-bipolar electrosurgical cutting device, greatly reduces or eliminates these potential problems.

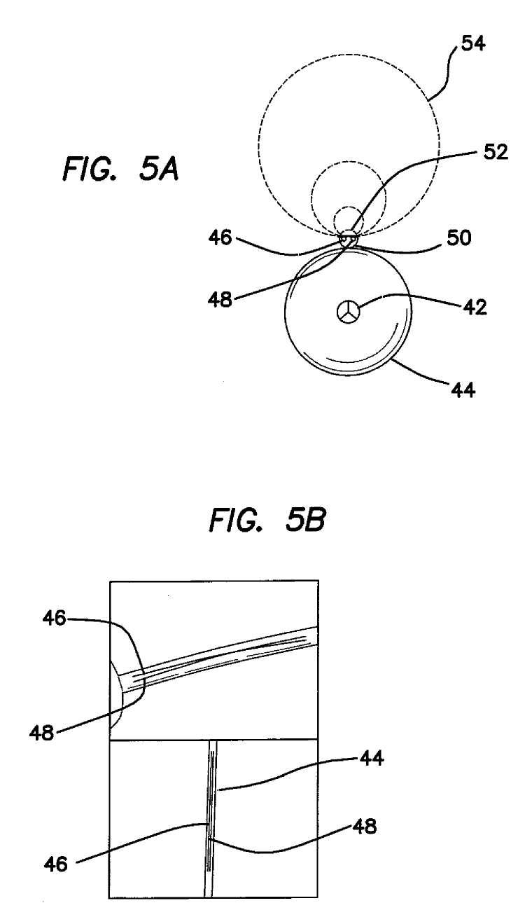

A first embodiment of the current invention is shown schematically in FIG. 5a, describing a bipolar, two cutting wire arrangement. A catheter body 42 with a three-lumen configuration is employed with a non-distensible balloon 44. Materials suitable for the balloon include low density polyethylene (LDPE), polyetheretherketones (PEEK), polyether block amides (PEBA), polytetrafluoroethylene (PTFE), nylon 11, nylon 12, and other similar compounds, as will be appreciated by those skilled in the art. Non-distensible balloons made from materials having melting temperatures below about 180° C. may be covered by a second balloon composed of a high melting temperature (greater than about 180° C.) material, such as silicone, to prevent damage to the underlying non-distensible balloons during the heating process.

Some materials exhibit both the desired non-distensible qualities and high-melting-temperatures and can be used in balloons without a secondary covering. Examples of such materials include, but are not limited to, nylon 11 and nylon 12, and other non-distensible balloon materials having a melting temperature greater than about 180° C.

In one embodiment, a balloon composed of LDPE, PEBA, PEEK or nylon 12 and having a wall thickness of approximately 0.0015-inch is used with a silicone balloon having a wall thickness of approximately 0.0025-inch (in the inflated state). In another embodiment, a balloon composed of nylon 11 or nylon 12 and having a wall thickness from about 0.0015-inch to about 0.005-inch is used without a silicone balloon covering. The inflated outer balloon(s) diameter typically is from about 24 French to about 30 French.

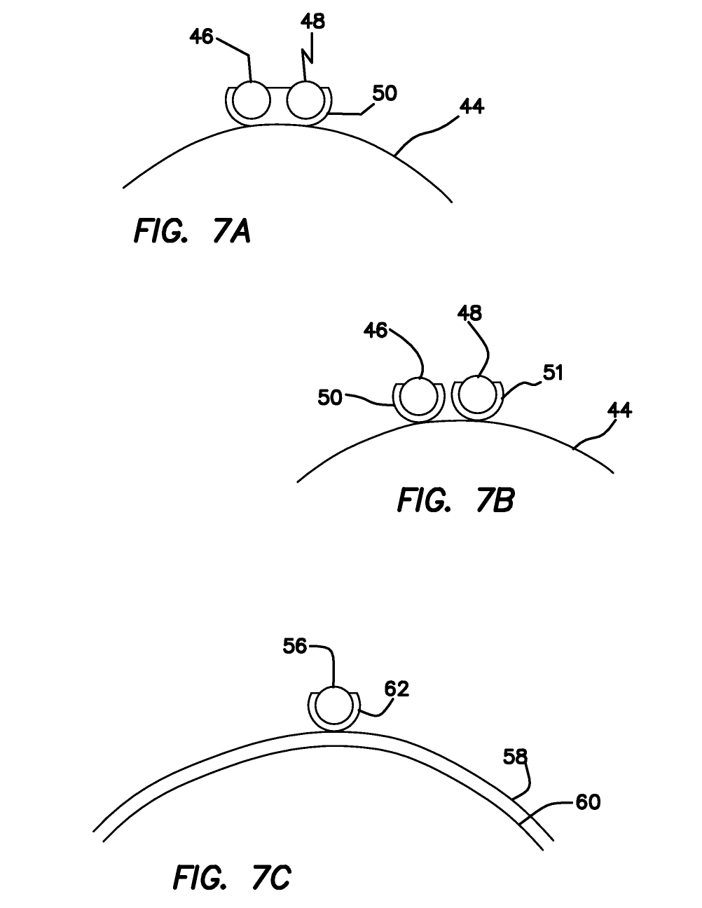

While one lumen carries a guide wire, and a second lumen provides the channel for the insufflation fluid, the third lumen carries two electrical cutting wires 46 and 48, imbedded in an electrical insulation sleeve 50. Alternatively, each wire may be separately imbedded in an electrical insulation sleeve. In either case, the portions of the insulation sleeve facing away from the balloon(s) are cut or sliced or otherwise open to leave the wire(s) exposed. FIG. 5b shows the two cutting wires on the outside of the inflated balloon 44. FIGS. 7a and b show close-ups of the electrical cutting wires imbedded in a single insulation sleeve (7a) or in separate insulation sleeves (7b).

As with the monopolar catheter assembly, the material used for the cutting wires in the bipolar catheter assembly can be any kind of materials currently used for electrosurgical cutting, such as, for example, stainless steel or tungsten. In one embodiment, 0.010-inch to 0.015-inch stainless steel cutting wire is used with approximately 0.0025-inch fluorinated ethylene propylene (FEP) insulation material.

In this bipolar arrangement, the electrical current is flowing from one cutting wire—through the tissue—to the second wire. With this configuration, the return electrode patch applied to the skin of the patient when using a monopolar device is not required. Moreover, the bipolar configuration creates its own spark-gap between the two wire electrodes. The current density in the tissue immediate to the exposed cutting wires 46 and 48 is exposed to a high current (and energy) density 52, and is quickly cut. Unlike the monopolar case, the electrical current in the bipolar case does not travel through large volumes of tissue, and instead is restricted to the tissue in very close proximity to the cutting site. The electrical current between the two cutting wires actually “spills” over into neighboring tissue, following the electrical field generated within the tissue. This is illustrated in FIG. 5 by the electrical field lines 54.

Because electrical energy is directed only entirely into the tissue between the two wires, resulting in the high current (and energy) density that results in cutting, and because the electricity does not have to travel through large volumes of bulk tissue, the bipolar configuration shown in FIG. 5 requires less total power to achieve the same cutting effect as the monopolar configuration. At the same time, the absence of current traveling through large volumes of bulk tissue (outside the view of the surgeon), eliminates the risk of electrical burns through constricting tissue elements or delaminating return electrode patches.

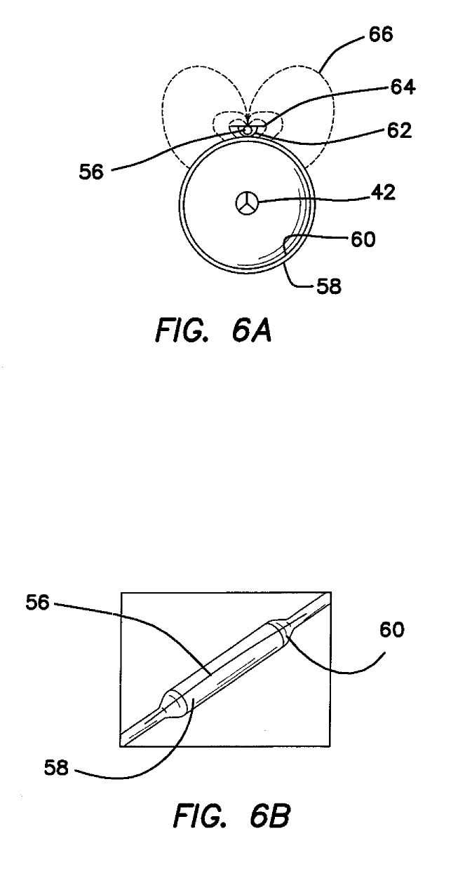

Another embodiment of the present invention is depicted in FIG. 6, showing a quasi-bipolar cutting arrangement. Here, the cutting wire 56 is again positioned on the outside balloon surface, as in the monopolar configuration, while a second electrode 58 is arranged on the outside surface of the entire balloon surface 60. When inserting the balloon arrangement into a restricted body cavity or orifice, dilatation of the balloon leads to electrical contact between both the exposed section of the cutting wire 56 and the return electrode 58 with the tissue. The electrical insulation on the cutting wire 62 prevents immediate contact and electrical shorting between the cutting wire and the return electrode.

When applying electrical power to the arrangement shown in FIG. 6, electrical current flows from the exposed section of the cutting wire 56 into the contacting tissue. Because of the small contact area, the current density close to the cutting wire is high (shown schematically in FIG. 6 at 64), leading to electrosurgical cutting of the constricted tissue. From here, the electrical current follows the electrical field lines 66 toward the return electrode 58, which is the entire outer balloon surface 60. Because of the relatively large contact area, the current density disperses through the entire tissue in contact with the return electrode. Similar to the return electrode patch in the monopolar configuration, the relatively large area of the return electrode causes dispersion of the electrical current, reducing the deposited energy density to an insignificant level.

In addition, as electrical energy is essentially directed only into the tissue contacting the cutting wire, and since the electricity does not have to travel through large volumes of bulk tissue, the bipolar configuration shown in FIG. 6 will again call for less total power to achieve the same cutting effect as the monopolar configuration. At the same time, the absence of current traveling through large volumes of bulk tissue, outside the view of the surgeon, eliminates the risk of electrical burns through constricting tissue elements or delaminating return electrode patches.

Other embodiments of the present invention appropriate for an arrangement as shown in FIG. 6 would involve a metallized balloon, generated by vacuum-coating or sputter-coating a non-distensible balloon with a metal or metal alloy.

Other embodiments of the present invention can include any provision of material on the outside surface of the balloon, making it electrically conductive (such as metallized pastes, indium tin oxide (ITO), etc.) Yet another embodiment of the present invention can include a balloon made of a non-distensible, electrically conductive polymer.

Although the present invention has been described in certain specific aspects, many additional modifications and variations would be apparent to those skilled in the art. It is therefore to be understood that the present invention may be practiced otherwise than specifically described, including various changes in the size, shape and materials, without departing from the scope and spirit of the present invention. Thus, embodiments of the present invention should be considered in all respects as illustrative and not restrictive. Also, all the examples provided throughout the entire description should be considered in all respects as illustrative and not restrictive. The scope of the invention is, therefore, indicated by the following claims rather than by the foregoing description. All changes, modifications, and variations coming within the meaning and range of equivalency of the claims are to be considered within their scope.

We claim:

1. A bipolar dilation-and-cutting catheter assembly adapted for insertion into a body conduit of a patient, comprising:
an elongate tubular body having an axis and a distal end carrying a generally cylindrical radially dilatable member adapted to be positioned longitudinally in a body conduit and having properties for dilating generally radially of the tubular body;
at least two wires carried by the tubular body exteriorly of the dilatable member, the wires disposed longitudinally of the tubular body and movable radially in a plane including the axis of the tubular body;
means for dilating the dilatable member to exert dilation forces on the body conduit and to move the wires to a position adjacent to the tissue of the body conduit; and
means for activating the wires to create an incision in the tissue.

2. The assembly recited in claim 1, wherein the dilatable member is an inflatable balloon that is adapted to be connected to a source of inflation fluid.

3. The assembly recited in claim 2, wherein the balloon is comprised of a non-distensible material.

4. The assembly recited in claim 3, wherein non-distensible material is selected from the group consisting of low density polyethylene (LDPE), polyetheretherketone (PEEK), polyether block amide (PEBA), nylon 11, nylon 12, and mixtures thereof.

5. The assembly recited in claim 2, further comprising a second balloon disposed around the inflatable balloon.

6. The assembly recited in claim 5, wherein the second balloon is comprised of a high melting temperature material.

7. The assembly recited in claim 6, wherein the second balloon is comprised of silicone.

8. The assembly recited in claim 2, wherein the balloon is comprised of a high-melting-temperature, non-distensible material.

9. The assembly recited in claim 1, wherein the activating means comprises a generator having electrical power to create a current density in the tissue proximate to the wires, the electrical power being sufficient to cut the tissue.

10. A bipolar dilation catheter assembly adapted for insertion into a body conduit of a patient, comprising:
an elongate tubular body having an axis and a distal end carrying a generally cylindrical inflatable balloon that is adapted to be connected to a source of inflation fluid and that is adapted to be positioned longitudinally in a body conduit and having properties for dilating generally radially of the tubular body;
a pair of wires carried by the tubular body exteriorly of the inflatable balloon, the wires disposed longitudinally of the tubular body and movable radially in a plane including the axis of the tubular body, wherein at least one of the wires is adapted to connect to a generator having electrical power to create a current density in the tissue proximate to the wires, the electrical power being sufficient to cut the tissue.

11. A quasi-bipolar dilation-and-cutting catheter assembly adapted for insertion into a body conduit of a patient, comprising:
an elongate tubular body having an axis and a distal end carrying a generally cylindrical radially dilatable member adapted to be positioned longitudinally in a body conduit and having properties for dilating generally radially of the tubular body;
a first electrode, comprising a wire carried by the tubular body exteriorly of the dilatable member, the wire disposed longitudinally of the tubular body and movable radially in a plane including the axis of the tubular body;
a second electrode disposed exteriorly around the surface of the dilatable member;
means for dilating the dilatable member to exert dilation forces on the body conduit and to move the first and second electrodes to a position adjacent to the tissue of the body conduit; and
means for activating the electrodes to create an incision in the living tissue.

12. The assembly recited in claim 11, wherein the dilatable member is an inflatable balloon that is adapted to be connected to a source of inflation fluid.

13. The assembly recited in claim 12, wherein the balloon is comprised of a non-distensible material.

14. The assembly recited in claim 13, wherein non-distensible material is selected from the group consisting of low density polyethylene (LDPE), polyetheretherketone (PEEK), polyether block amide (PEBA), nylon 11, nylon 12, and mixtures thereof.

15. The assembly recited in claim 12, wherein the dilatable member further comprises a second balloon disposed around the inflatable balloon.

16. The assembly recited in claim 15, wherein the second balloon comprises a high-melting-temperature material.

17. The assembly recited in claim 16, wherein the high-melting-temperature material is silicone.

18. The assembly recited in claim 11, wherein the second electrode is an integral component of the dilatable member.

19. The assembly recited in claim 18 wherein the second electrode comprises an electrically conductive polymer.

20. The assembly recited in claim 18 wherein the second electrode comprises a metallized balloon.

21. The assembly recited in claim 11, wherein the second electrode is permanently affixed to the exterior surface of the dilatable member.

22. The assembly recited in claim 20, wherein the second electrode comprises a material selected from the group consisting of metallized paste, indium tin oxide, electrically conductive polymer, and mixtures thereof.

23. The assembly recited in claim 11, wherein the activating means comprises a generator having electrical power to create a current density in the tissue proximate to the electrodes, the electrical power being sufficient to cut the tissue.

24. A quasi-bipolar dilation catheter assembly adapted for insertion into a body conduit of a patient, comprising:
an elongate tubular body having an axis and a distal end carrying a generally cylindrical inflatable balloon that is adapted to be connected to a source of inflation fluid and that is adapted to be positioned longitudinally in a body conduit and having properties for dilating generally radially of the tubular body;
an electrode disposed around the outside surface of the balloon; and
a wire carried by the tubular body exteriorly of the balloon, the wire disposed longitudinally of the tubular body and movable radially in a plane including the axis of the tubular body, wherein the wire is adapted to connect to a generator having electrical power to create a current density in the tissue proximate to the wire, the electrical power being sufficient to cut the tissue.

25. The quasi-bipolar dilation catheter assembly recited in claim 24, wherein the second electrode comprises a material selected from the group consisting of metallized paste, indium tin oxide, electrically conductive polymer, and mixtures thereof.

26. The quasi-bipolar dilation catheter assembly recited in claim 24, wherein the electrode comprises an electrically conductive polymer.

27. An apparatus for cutting a body conduit, comprising:
a supporting structure having an outer surface;
a first electrode having a first portion disposed in a fixed relationship with the supporting structure and a second portion disposed outwardly of the outer surface in a movable relationship with the supporting structure;
moving means disposed between the supporting structure and the second portion of the first electrode for moving the second portion of the first electrode into proximity with the tissue to be cut;
a second electrode, disposed on the exterior of the moving means; and
activating means for electrically activating the electrodes to cut the body conduit.

28. The apparatus recited in claim 27, wherein the first electrode is a wire adapted to connect to a generator having electrical power to create a current density in the tissue proximate to the wire sufficient to cut the tissue.

29. The apparatus recited in claim 27, wherein the second electrode is a wire having a first portion disposed in a fixed relationship with the supporting structure and a second portion disposed outwardly of the outer surface in a movable relationship with the supporting structure.

30. The apparatus recited in claim 27, wherein the supporting structure defines a lumen and the moving means comprises a balloon inflatable through the lumen, the balloon having first portions fixed to the supporting structure and second portions movable relative to the supporting structure, the second portions of the balloon being disposed between the supporting structure and the second portion of the first electrode to move the first electrode into proximity with the body conduit when the balloon is inflated.

31. The apparatus recited in claim 30, wherein the second electrode is an integral component of the balloon.

32. The assembly recited in claim 31, wherein the second electrode comprises an electrically conductive polymer.

33. The assembly recited in claim 31, wherein the second electrode comprises a metallized balloon.

34. The assembly recited in claim 30, wherein the second electrode is permanently affixed to the exterior surface of the balloon.

35. The assembly recited in claim 34, wherein the second electrode comprises a material selected from the group consisting of metallized paste, indium tin oxide, electrically conductive polymer, and mixtures thereof.
